(19) United States
(12) Patent Application Publication
(10) Pub. No.: US 2008/0268048 A1
(43) Pub. Date: Oct. 30, 2008

(54) PHARMACEUTICAL COMPOSITION FOR CONTRACEPTION AND FOR REDUCING THE RISK OF CONGENITAL ABNORMALITIES

(76) Inventor: Claus Claussen, Jena (DE)

Correspondence Address: STRIKER, STRIKER & STENBY, 103 EAST NECK ROAD, HUNTINGTON, NY, 11743, US

(21) Appl. No.: 11/773,037

(22) Filed: Jul. 3, 2007

(30) Foreign Application Priority Data
Jul. 6, 2006 (EP) 06 014 002.7
Aug. 14, 2006 (EP) 06 016 950.5

(51) Int. Cl. A61K 31/56; A61K 31/724; A61K 31/717; A61K 9/28; A61K 9/14; A61P 15/18

(52) U.S. Cl. 424/474; 514/170; 514/58; 514/57; 424/489

(57) ABSTRACT

The pharmaceutical composition for oral contraception and for reducing the risk of congenital abnormalities contains, in a daily dosage unit, 2.0 mg or 1.5 mg of 17α-cyanomethyl-17β-hydroxyoestra-4,9-dien-3-one (dienogest), 0.015 mg of 17α-ethynyloestradiol (ethynyloestradiol), and (6S)-5-methyl-tetrahydrofolate, preferably as the calcium salt of (6S)-5-methyltetrahydrofolic acid (metafolin), together with one or more pharmaceutically acceptable auxiliaries/carriers. A kit for oral contraception contains 21 daily dosage units of the pharmaceutical composition and 7 daily dosage units solely containing (6S)-5-methyltetrahydrofolate, preferably in the form of metafolin.

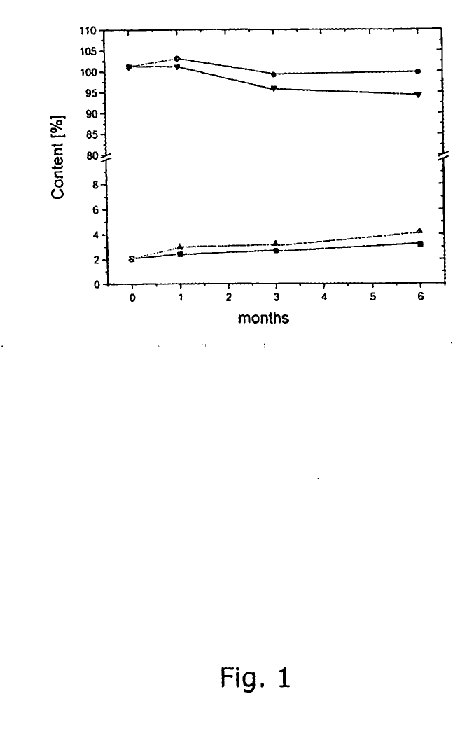

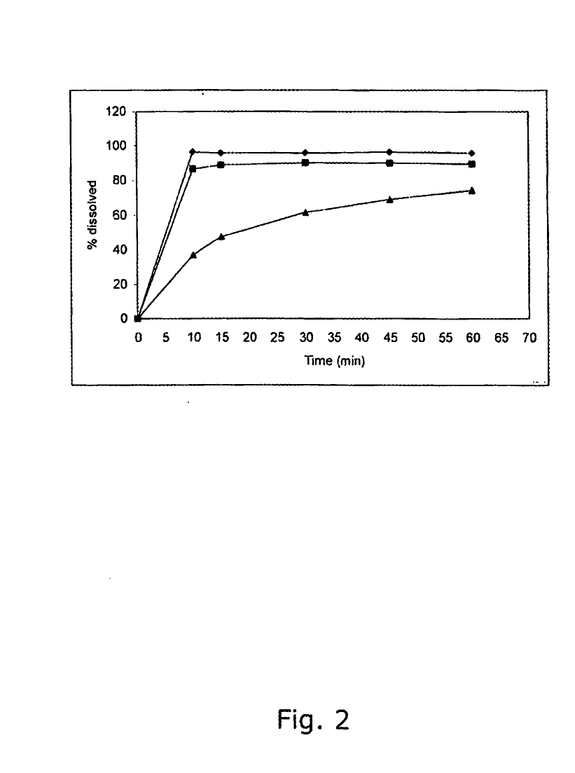

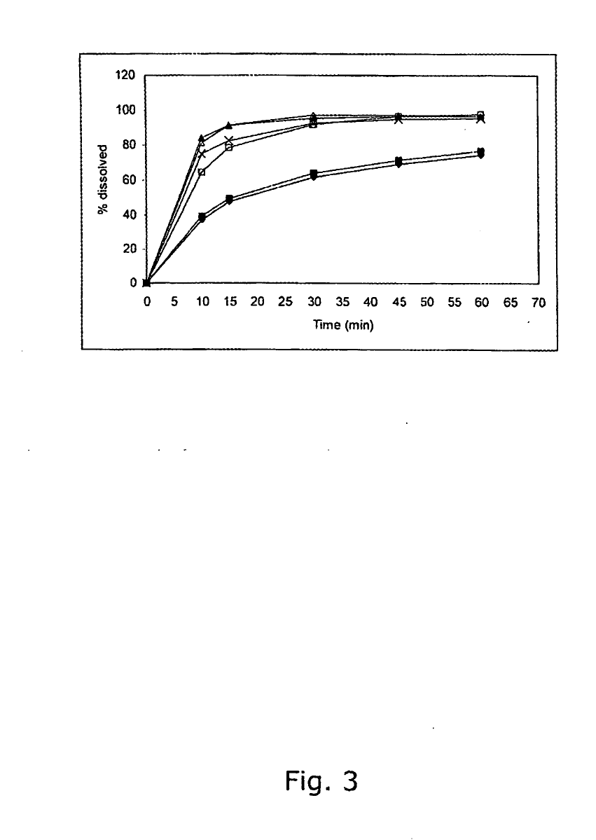

### CROSS-REFERENCE

The invention described and claimed in the present specification is based on the disclosures of European Patent Applications EP 06 016 950.5 filed Aug. 14, 2006 and EP 06 014 002.7 filed Jul. 6, 2006 in Europe. The foregoing European Patent Applications provide the basis for a claim of priority for the invention disclosed herein below under 35 U.S.C. 119 and their subject matter is explicitly incorporated herein by reference.

### BACKGROUND OF THE INVENTION

1. The Field of the Invention

The subject matter of the invention includes a pharmaceutical composition for contraception and for reducing the risk of congenital abnormalities, which comprises, in a daily dose,
2.0 mg of 17α-cyanomethyl-17β-hydroxyoestra-4,9-dien-3-one (dienogest) and 0.015 mg of 17α-ethynyloestradiol (ethynyloestradiol) and (6S)-5-methyltetrahydrofolate ((6S)-5-MTHF) or
1.5 mg of dienogest and 0.015 mg of ethynyloestradiol and (6S)-5-methyl-tetrahydrofolate ((6S)-5-MTHF);
together with one or more pharmaceutically acceptable auxiliaries/carriers.

The invention also includes a kit, which comprises 21 daily dose units of the active compound combination described above and 7 daily dose units with (6S)-5-MTHF.

The invention also relates to a tablet, preferably a film tablet, with the active compound combination described above. The tablet core comprises a proportion of the dienogest, no (6S)-5-MTHF, a part of or the total content of the (6S)-5-MTHF, and the film coating comprises the other portion of the dienogest, no (6S)-5-MTHF, a part of or the total content of the (6S)-5-MTHF and the total content of the ethynyloestradiol.

2. Related Art

Oral contraceptive agents comprising a gestagen component and an oestrogen component came onto the market for the first time in the early 1960s. Three essential properties characterize the profile of the “contraceptive pill”: contraceptive reliability, very good cycle control and a minimum of side effects. Since the introduction of hormonal contraceptives, research has been directed toward creating medicament forms which, with the same good contraceptive reliability and cycle control, reduce undesirable side effects, such as, for example, arterial and venous thromboses and reduce their influence on carbohydrate and fat metabolism—caused by a higher content of gestagen and oestrogen than is necessary for contraception.

WO 98/004269 discloses, inter alia, oral administration of a combination of 250 μg-4 mg of dienogest and 10 μg-20 μg of ethynyloestradiol for contraception. In order to achieve the considerable reduction in the total contraceptive steroid administered per cycle, while retaining a good cycle control, the low-dose gestagen/oestrogen combination is administered for 23 to 25 days of a 28-day menstrual cycle. However, no results and information, which demonstrate that the inventive idea is also successful and what type of release of the steroids is aimed at, are disclosed in that patent specification.

Folic acid, also called pteroyl-mono-glutamic acid, N-(4-(((2-amino-1,4-dihydro-4-oxo-6-pteridinyl)methyl)-amino)benzoyl)glutamic acid (empirical formula: C₁₉H₁₉N₇O₆), folinic acid, is a heat- and light-sensitive, water-soluble vitamin from the vitamin B complex (vitamin B₉).

It is known that folates are predominantly present in food as pteroylpoly-glutamates. After food intake these are first hydrolysed in the mucosa cells to give pteroylmonoglutamates, and are then chiefly absorbed in the intestine by active transport.

In the liver, the predominantly non-methylated folates are converted into methylated folates and are chiefly transported further to cells as 5-methyl-tetrahydrofolate (5-MTHF) bonded to albumin and α-macroglobulin, taken up there, demethylated and converted into the polyglutamate form.

The amino acid homocysteine and an enzyme, which requires vitamin B₁₂ as a coenzyme, are involved in the demethylation.

It is furthermore known that losses in the folate content of foodstuffs can arise due to the preparation (cooking) and storage. It is moreover known that intensive UV radiation impinging on the human skin reduces folic acid in the body. Pale-skinned humans are particularly affected in this context.

If the supply of folate and/or vitamin B₁₂ is inadequate, homocysteine metabolism is impeded, and as a consequence the concentration of homocysteine in the blood can rise. The concentration of homocysteine in the blood can accordingly be used as an indicator of the folate content.

The state of hyperhomocysteinaemia is defined according to Malinow, M R et al, Homocyst(e)ine, diet, and cardiovascular disease, Statement for healthcare professionals from the Nutrition Committee, American Heart Association, Circulation 99, 178-182, 1999 by the following concentration in the plasma: 16-30 μmol/l (moderate); 31-100 μmol/l (medium); >100 μmol/l (severe).

A concentration above 10 μmol/l is regarded as critical and from 12 μmol/l action is required.

In addition to the deficiency of folate and vitamin B₁₂, however, enzyme defects can also affect the increase in the homocysteine concentration. The connection between increased homocysteine concentrations in the blood and vascular diseases has been debated for some time, for example also as a risk factor for cardiovascular diseases.

It is also debated whether folic acid/folate can protect against malignant diseases because of its significance for DNA methylation and DNA strand stability.

It is also known that an inadequate folate status in pregnancy can lead to congenital abnormalities, such as, for example, congenital heart defects, congenital abnormalities of the urinary tract, an acute lymphoblastic leukaemia, cleft lip, jaw and palate or abnormalities of the central nervous system, such as medullary defects (spina bifida or anencephaly).

The Deutsche Gesellschaft für Ernährung e.V. thus recommends a daily dose of 400 μg of folic acid in principle, 600 μg for pregnant women and 600 μg for breastfeeding mothers. This is a global statement. Deficiencies in vitamin B₁₂ and folate deficiency show identical changes in the blood count. Folate deficiency can be compensated by administration of folate/folic acid, but the deficiency in vitamin B₁₂ is not indicated. There is therefore the danger of a masked vitamin B₁₂ deficiency.

The patent specification U.S. Pat. No. 6,190,693 B1 discloses a method for administration of folic acid simultaneously with a conventional oral contraceptive for use as an oral contraceptive. The publication WO 2003/070255 discloses an oral contraceptive, and a kit for oral, hormonal contraception, which comprises oestrogens and/or gestagens, tetrahydrofolates and necessarily vitamin B₁₂ or optionally vitamin B₆.

WO 2005/115349 discloses a presentation form for hormonal contraception with hormone-containing daily units and hormone-free daily units, the hormone-containing daily units comprising up to at most 200 μg of folic acid and the hormone-free daily units comprising greater than 200 μg of folic acid.

The patent specification EP 0 898 965 claims the use of 5-methyl-(6S)-tetrahydrofolic acid or pharmaceutically acceptable salts thereof for preventing medullary defects.

The patent specification EP 1 044 975 discloses crystalline salts of 5-methyl-(6R,S)—, -(6S)-and-(6R)-tetrahydrofolic acid and their use as constituents of a foodstuff supplement.

### SUMMARY OF THE INVENTION

It is an object of the present invention to provide a pharmaceutical composition based on dienogest and ethynyloestradiol, the steroid dosage of which is reduced and which simultaneously reduces the risk of congenital abnormalities after the onset of pregnancy.

This object according to the invention is attained by a pharmaceutical composition for contraception and for reducing the risk of congenital abnormalities, comprising in a daily dose of 2.0 mg of 17α-cyanomethyl-17β-hydroxyoestra-4,9-dien-3-one (dienogest), 0.015 mg of 17α-ethynyl-estradiol (ethynyloestradiol), and (6S)-5-methyltetrahydrofolate ((6S)-5-MTHF); or 1.5 mg of dienogest, 0.015 mg of ethynyloestradiol, and (6S)-5-methyl-tetrahydrofolate ((6S)-5-MTHF); together with one or more pharmaceutically acceptable auxiliaries/carriers.

(6S)-5-MTHF or (6S)-5-methyltetrahydrofolic acid can also be called 5-methyl-(6S)-tetrahydrofolates or 5-methyl-(6S)-tetrahydrofolic acid.

In the pharmaceutical composition according to the invention, the free acid form and pharmaceutically acceptable salts and modifications of (6S)-5-methyl-tetrahydrofolic acid (N-[4-[[(2-amino-1,4,5,6,7,8-hexahydro-4-oxo-5-methyl-(6S)-pteridinyl)methyl]amino]benzoyl]-L-glutamic acid) are called (6S)-5-MTHF. Pharmaceutically acceptable salts should be both pharmacologically and pharmaceutically acceptable. Such pharmacologically and pharmaceutically acceptable salts can be alkali metal or alkaline earth metal salts, preferably sodium, potassium, magnesium or calcium salts. The calcium salt of (6S)-5-methyltetrahydrofolic acid (metafolin) can also be incorporated in differently suitable crystal forms.

The crystalline calcium salt of (6S)-5-methyltetrahydrofolic acid (metafolin) is advantageously employed according to the invention as (6S)-5-MTHF. According to the invention, the calcium salt of (6S)-5-methyltetrahydrofolic acid (metafolin) is used in a dosage of from 0.4 to 1 mg, preferably 451 μg. Similarly, above-identified calcium salt can be used as a racemate in a dosage of 100 to 800 μg or incorporated in a microencapsulated form.

Alternatively, (6R)-5-methyltetra-hydrofolate can also be employed for (6S)-5-MTHF in about twice the dosage.

The object is also achieved according to the invention with a tablet, preferably a film tablet with the active compound combination described above. The tablet core comprises a part of the dienogest, no (6S)-5-MTHF or a part of or the total content of the (6S)-5-MTHF, and the film coating comprises the remaining part of the dienogest, no (6S)-5-MTHF or a part of or the total content of the (6S)-5-MTHF and the total content of ethynyloestradiol. In other words, the film tablet has a tablet core with a part of the total content of dienogest, which is to be released in a retarded manner, and a film coating with the rest of the total content of dienogest, which is to be released in a non-retarded manner (rapidly), and a total content, which is to be released in a non-retarded manner (rapidly), of ethynyloestradiol—and in the tablet core a part of the total content of (6S)-5-MTHF, which is to be released in a retarded manner, and in the film coating a remaining part of the total content of (6S)-5-MTHF, which is to be released in a non-retarded manner (rapidly), or in the tablet core a total content of (6S)-5-MTHF, which is to be released in a retarded manner, or in the film coating a total content of (6S)-5-MTHF, which is to be released in a non-retarded manner (rapidly). The part of the dienogest and the part of the (6S)-5-MTHF are dissolved out of the tablet core at least to the extent of 10%, preferably to the extent of 30%, or optionally the total content of (6S)-5-MTHF, in a virtually completely retarded manner after longer than 30 minutes, as is determined with the dissolution test using water at 37° C. as the dissolution medium and 50 rpm as the stirring speed.

The determination is carried out by means of a rotating basket apparatus using 1000 ml of water, in accordance with Ph.Eur.

The part of the dienogest, like the total content of ethynyloestradiol and like the total content of (6S)-5-MTHF, is dissolved out from the film coating to the extent of at least 75% in not more than 45 min, preferably to the extent of 70% in 30 min, as is determined with the dissolution test using water at 37° C. as the dissolution medium and 50 rpm as the stirring speed. It is also conceivable that in the second phase, together with the part of the dienogest, the part of the total content of ethynyloestradiol is released in a delayed manner, preferably from the tablet core.

The object is also achieved according to the invention by a kit which comprises 21 daily dose units of 2.0 or 1.5 mg of dienogest and in each case 0.015 mg of 17α-ethynyloestradiol and (6S)-5-MTHF, preferably 451 μg of the calcium salt of 5-methyl-(6S)-tetrahydrofolic acid (metafolin), in each daily dose unit and 7 daily dose units with (6S)-5-MTHF alone without the steroid combination, preferably 451 μg of metafolin in each daily dose unit. Alternatively, the object can also be achieved by a kit, which comprises 22 to 24 daily dose units of 2.0 or 1.5 mg of dienogest and in each case 0.015 mg of 17α-ethynyloestradiol and (6S)-5-MTHF and 4 to 6 daily dose units with (6S)-5-MTHF alone without the steroid combination, preferably as (6S)-5-MTHF 451 μg of the calcium salt of 5-methyl-(6S)-tetrahydrofolic acid (metafolin) in each daily dose unit; or which comprises 21 to 24 daily dose units of the active compound combination described above and 4 to 7 daily dose units of placebo, i.e. without hormones; or which comprises 21 to 24 daily dose units of 2.0 or 1.5 mg of dienogest and in each case 0.015 mg of 17α-ethynyloestradiol and 4 to 7 daily dose units with (6S)-5-MTHF alone without the steroid combination, preferably 451 μg of the calcium salt of 5-methyl-(6S)-tetrahydrofolic acid (metafolin), in each daily dose unit. The object of the invention is also achieved according to the invention by a method of using the active compound combination described, namely the combination of 2.0 mg or 1.5 ml of dienogest, and in each case 0.015 mg of ethynyloestradiol and (6S)-5-MTHF, together with one or more pharmaceutically acceptable auxiliaries/carriers for preparation of a pharmaceutical composition for reducing the risk of congenital abnormalities in pregnancy which are related to folate deficiency. Ethynyloestradiol-beta-cyclodextrin complex can also be employed as ethynyloestradiol. In the case of the use of ethynyloestradiol-beta-cyclodextrin complex (1:2), not more than or approximately ten times the amount is to be employed.

It may also prove to be advantageous in this context if the intake of (6S)-5-MTHF, preferably 451 μg of the calcium salt of (6S)-5-methyltetrahydrofolic acid (metafolin), in each daily dose unit, takes place optionally until the onset of pregnancy, during pregnancy and during the breastfeeding period (here optionally also in a higher dosage).

### EMBODIMENT EXAMPLES

### Example 1

Tablets having the following composition are prepared:

| Core: | |
|---|---|
| Dienogest | 2.000 mg or 1.500 mg |
| Ethynyloestradiol | 0.015 mg |
| Metafolin | 0.451 mg |
| Lactose monohydrate | 28.720 mg |
| Maize starch | 15.000 mg |
| Maltodextrin | 3.750 mg |
| Magnesium stearate | 0.500 mg |

An ethynyloestradiol-beta-cyclodextrin complex can also be employed as the ethynyloestradiol. In the case where the ethynyloestradiol-beta-cyclodextrin complex (1:2) is used, not more than or approximately ten times the amount is to be employed.

All the substances are mixed and granulated in a suitable manner. The absorption of the metafolin, after conclusion of the granulation process, renewed mixing, tablet-making and optionally film-coating take place.

### Example 2

Blood is taken from healthy young women of childbearing age at an interval of 8 weeks and the erythrocyte folate level is determined with a validated microbiological, immunological or instrumental (e.g. HPLC, LC-MS/MS) method or a suitable combination of these methods.

Approx. 8 weeks after the first blood sample (screening phase), 451 μg of the calcium salt of 5-methyl-(6S)-tetrahydrofolate are administered daily over a period of approx. 40 weeks, or alternatively 2 or 1.5 mg of dienogest, 15 μg of ethynyloestradiol and 451 μg of the calcium salt of 5-methyl-(6S)-tetrahydrofolic acid are administered simultaneously in each case in the first 21 days of the particular cycle (metafolin) (tablet according to embodiment example 1). In an immediately subsequent phase, the administration of 451 μg of the calcium salt of 5-methyl-(6S)-tetrahydrofolic acid in the form of a tablet is continued for 7 days. 2.0 or 1.5 mg of dienogest, 15 μg of ethynyloestradiol and 451 μg of metafolin are administered again for a further 21 days (second cycle) and only 451 μg of metafolin are administered for a further 7 days, and so on.

We claim:

1. A pharmaceutical composition for contraception and for reducing risk of congenital abnormalities, said pharmaceutical composition comprising, in a daily dosage unit,
2.0 mg of 17α-cyanomethyl-17β-hydroxyoestra-4,9-dien-3-one, 0.015 mg of 17α-ethynyloestradiol, (6S)-5-methyltetrahydrofolate, and one or more pharmaceutically acceptable auxiliaries and/or carriers; or
1.5 mg of said 17α-cyanomethyl-17β-hydroxyoestra-4,9-dien-3-one, 0.015 mg of said ethynyloestradiol, said (6S)-5-methyl-tetrahydrofolate, and one or more of said pharmaceutically acceptable auxiliaries and/or carriers.

2. The pharmaceutical composition as defined in claim 1, in which the (6S)-5-methyltetrahydrofolic acid is provided in the form of a calcium salt of the (6S)-5-methyltetrahydrofolic acid in one or more different crystal forms.

3. The pharmaceutical composition as defined in claim 1, in which the (6S)-5-methyltetrahydrofolic acid is provided in the form of a crystalline calcium salt of the (6S)-5-methyltetrahydrofolic acid in one or more different crystal forms or in which the (6S)-5-methyltetrahydrofolic acid is provided in the form of a microencapsulated calcium salt of said (6S)-5-methyl-tetrahydrofolic acid in one or more different crystal forms.

4. The pharmaceutical composition as defined in claim 1, containing a daily dosage of from 0.4 to 1 mg of said (6S)-5-methyltetrahydrofolate.

5. The pharmaceutical composition as defined in claim 1, comprising a daily dose of 451 μg of a calcium salt of said (6S)-5-methyltetrahydrofolic acid.

6. A kit containing
21 daily dosage units of a pharmaceutical composition; and
daily dosage units each containing (6S)-5-methyltetrahydrofolate; in which each of the daily dosage units of the pharmaceutical composition comprises
2.0 mg of 17α-cyanomethyl-17β-hydroxyoestra-4,9-dien-3-one, 0.015 mg of 17α-ethynyloestradiol, said (6S)-5-methyltetrahydrofolate, and one or more pharmaceutically acceptable auxiliaries and/or carriers; or
1.5 mg of said 17α-cyanomethyl-17β-hydroxyoestra-4,9-dien-3-one, 0.015 mg of said ethynyloestradiol, said (6S)-5-methyl-tetrahydrofolate, and one or more of said pharmaceutically acceptable auxiliaries and/or carriers.

7. The kit as defined in claim 6, in which each of the daily dosage units of the pharmaceutical composition and of the (6S)-5-methyl-tetrahydrofolate comprises 451 μg of a calcium salt of the (6S)-5-methyltetrahydrofolic acid.

8. A method of preparing a pharmaceutical composition for reducing a risk of congenital abnormalities in pregnancy, said method comprising using 2.0 mg of dienogest, 0.015 mg of ethynyloestradiol, (6S)-5-methyltetrahydrofolate, and one or more pharmaceutically acceptable auxiliaries/carriers; or using 1.5 mg of said dienogest, 0.015 mg of said ethynyloestradiol, said (6S)-5-methyl-tetrahydrofolate, and one or more of said pharmaceutically acceptable auxiliaries/carriers.

9. The method as defined in claim 8, further comprising using 451 μg of a calcium salt of said (6S)-5-methyltetrahydrofolic acid, 2.0 or 1.5 mg of said dienogest and 0.015 mg of said ethynyloestradiol to prepare the pharmaceutical preparation.

10. A pharmaceutical composition comprising an active ingredient combination in which the active ingredient combination consists of 2.0 or 1.5 mg of dienogest, 0.015 mg of ethynyloestradiol, and (6S)-5-methyltetrahydrofolate, said pharmaceutical composition is in the form of a tablet, said tablet consists of a tablet core and a film coating around the tablet core, and the tablet core contains a part of the total content of the dienogest, which is to be released in a retarded manner, and the film coating contains a remaining part of the dienogest and a total content of the ethynyloestradiol, which are to be released in a non-retarded manner, and
either a total content of the (6S)-5-methyltetrahydrofolate is contained in the tablet core and is to be released in a retarded manner, or said total content of the (6S)-5-methyltetrahydrofolate is contained in the film coating and is to be released in a non-retarded manner tablet, or a part of the total content of the (6S)-5-methyltetrahydrofolate is contained in the tablet core and is to be released in a retarded manner and a remaining part of the total content of the (6S)-5-methyltetrahydrofolate is contained in the film coating and is to be released in a non-retarded manner.

11. The pharmaceutical preparation as defined in claim 10 and consisting of a film tablet.

12. A kit comprising
21 daily dosage units of a pharmaceutical composition; and
daily dosage units comprising (6S)-5-methyltetrahydrofolate;
in which said pharmaceutical composition comprises an active ingredient combination and the active ingredient combination consists of 2.0 or 1.5 mg of dienogest, 0.015 mg of ethynyloestradiol, and (6S)-5-methyltetrahydrofolate, said pharmaceutical composition is in the form of a tablet, said tablet consists of a tablet core and a film coating around the tablet core, and the tablet core contains a part of the total content of the dienogest, which is to be released in a retarded manner, and the film coating contains a remaining part of the dienogest and a total content of the ethynyloestradiol, which is to be released in a non-retarded manner, and
either a total content of the (6S)-5-methyltetrahydrofolate is contained in the tablet core and is to be released in a retarded manner, or said total content of the (6S)-5-methyltetrahydrofolate is contained in the film coating and is to be released in a non-retarded manner, or a part of the total content of the (6S)-5-methyltetrahydrofolate is contained in the tablet core and is to be released in a retarded manner and a remaining part of the total content of the (6S)-5-methyl-tetrahydrofolate is contained in the film coating and is to be released in a non-retarded manner.

13. The kit as defined in claim 12, in which each of said daily dosage units contain 451 μg of a calcium salt of said (6S)-5-methyltetrahydrofolic acid.

14. A method of using 2.0 mg of dienogest, 0.015 mg of ethynyloestradiol, (6S)-5-methyltetrahydrofolate, and one or more pharmaceutically acceptable auxiliaries/carriers; or 1.5 mg of said dienogest, 0.015 mg of said ethynyloestradiol, and said (6S)-5-methyltetrahydrofolate, and one or more of said pharmaceutically acceptable auxiliaries/carriers; for preparation of a pharmaceutical composition for reducing the risk of congenital abnormalities related to folate deficiency, after prior longer-term regular intake of said pharmaceutical composition.

15. The method as defined in claim 14, comprising using 451 μg of a calcium salt of said (6S)-5-methyltetrahydrofolic acid to provide said (6S)-5-methyl-tetrahydrofolic acid.
